(19) United States
(12) Patent Application Publication
(10) Pub. No.: US 2011/0139161 A1
(43) Pub. Date: Jun. 16, 2011

(54) Health Aid Device

(76) Inventor: Kuldeep Singh Dhanjal, Milpitas, CA (US)

(21) Appl. No.: 12/945,851

(22) Filed: Nov. 13, 2010

Related U.S. Application Data
(60) Provisional application No. 61/261,322, filed on Nov. 14, 2009.

(51) Int. Cl. A61F 6/04

(52) U.S. Cl. 128/844

(57) ABSTRACT

A tubular device for being disposed on a penis of a user is provided. The tubular device includes a proximal end to facilitate disposition of the tubular device on the penis and a distal end substantially opposite the proximal end. Further, the tubular device includes a wall defining a hollow tubular body for receiving the penis. The wall is such that it includes at least a first set of regions that have a first wall thickness and a second set of regions that have a second wall thickness. The first wall thickness is greater than the second wall thickness and at least one of an underside of the penis and underside of glans of the penis is not covered by the first set of regions or a portion thereof. Design of the tubular device ensures at least one of an effective increase in a girth of said penis, an effective increase in a length of said penis and an effective increase in time to ejaculation for said user of said device.

### FIELD OF INVENTION

The invention disclosed herein relates, in general, to health aid device. More specifically, the present invention relates to a health aid device to be worn over a penis.

### BACKGROUND

In human sexual relationship, responsiveness of females varies from that of males. Factors influencing such response result in the males tending to reach climax of sexual intercourse before the females.

For example, pre mature ejaculation (PME) affects significant proportion of men of all ages at some point of time. Most available statistics indicate that 30%-60% of all men have some degree of PME condition. Also, issues relating to perception about sizes (girth and/or length) of penis, psychologically affect various men, resulting in decreased confidence and performance during sexual intercourse. Such conditions often lead to a decrease in sexual pleasure for the females and often disrupt relationships amongst couples/partners.

In order to counter these and more such factors, various techniques and devices have been developed for use during sexual intercourse to enhance stimulation for the females and bring about parity in attaining climax of sexual intercourse.

Usually, PME is typically addressed by application of a desensitizing cream or usage of a condom that is coated with a desensitizing substance e.g. fluids/creams having benzocaine, Lidocaine and the like as an active ingredient. While this may prolong the time of sexual intercourse, but at same time can severely reduce pleasure experienced by the males during the sexual intercourse because they essentially numb the nerve endings. This often leads to men not achieving orgasm or ejaculation and sometimes even losing erection during the sexual intercourse. Also, if the chemical compound that is responsible for desensitizing comes in contact with vagina, it can also de-sensitize the vagina and significantly reduce stimulation for the females as well.

Further, for penis size enhancement, various devices and methods are available. For example, various herbal pills, ointments and oils are recommended for usage for an extended period of time. However, they do not have a proven track record and are mostly a marketing gimmick that appeals to human desire.

There are also various mechanical devices and exercises that apply varying degree of tensile forces on penis for an extended period of time to provide penis size enhancement. All of these are recommended for continuous use for extended period of time and are painful. Further, they are not practical for almost everyone who has a normal job and life. The success rate using these is often very low. Further, awareness relating to these methods or devices is low and in most cases, availability is restricted to online adult stores. Therefore majority of potential users are unable to have access these.

Further, concentric hollow sleeves (or sheaths) of a variety of materials like silicone or latex have been developed. These sleeves can be slid onto a penis leading to an addition to girth, due to the wall thickness of the sleeves. The increase in wall thickness is mostly uniform around the periphery. The sleeves can further have an extension that adds to a length of the penis when this sleeve is slid over the penis.

However, use of the sleeves has its disadvantages. For example, the sleeves have a concentric tubular construction and thickness of the sleeve that adds to girth of the penis, essentially acts as a wrap on the penis and significantly reduces stimulation of the penis during the sexual intercourse. Further, the sleeves are bulky and relatively expensive. Usually they are packaged and marketed only as adult toys through select online retailer that significantly limits number of potential customers. Also due to the nature of design, packaging and marketing approach most of the products are not yet socially commonplace and therefore not available in mainstream or mass retail market. Furthermore, usually the method of application of the sleeve onto the penis is often cumbersome.

Accordingly, in light of the above discussion, there is a need for an improvement in the current devices for enhancing stimulation for the females during sexual intercourse, in order to eliminate one or more drawbacks of the prior art.

### BRIEF DESCRIPTION OF FIGURES

The features of the present invention, which are believed to be novel, are set forth with particularity in the appended claims. The invention may best be understood by reference to the following description, taken in conjunction with the accompanying drawings. These drawings and the associated description are provided to illustrate some embodiments of the invention, and not to limit the scope of the invention.

FIG. 1a is a top elevation view of an exemplary device, in accordance with an embodiment of the present invention;

FIG. 1b is a partially cut-out perspective view of the exemplary device of FIG. 1a, illustrating a thick wall on one side and a thin wall on a diametrically opposite side, in accordance with an embodiment of the present invention;

FIG. 1c is a longitudinal cross sectional view of the device of FIG. 1b along the plane L1-L1, illustrating a relatively large wall thickness at this section of the device, in accordance with an embodiment of the present invention;

FIG. 1d is a longitudinal cross sectional view of the device of FIG. 1b along the plane K1-K1, illustrating a reduced wall thickness at this section of the device, in accordance with an embodiment of the present invention;

FIG. 1e is a cross sectional view of the device of FIG. 1b along the plane X1-X1, illustrating a thicker wall on one side and a thin wall on the diametrically opposite side, in accordance with an embodiment of the present invention;

FIG. 1f is a partially cut-out perspective view of the device of FIG. 1b along the plane X1-X1, illustrating a thicker wall on one side that is gradually thinning and becoming very thin on the diametrically opposite side, in accordance with an embodiment of the present invention;

FIG. 2a is a partially cut-out perspective of an exemplary device primarily targeted for addressing pre mature ejaculation (PME), in accordance with an embodiment of the present invention;

FIG. 2b is a cross-section view of the device of FIG. 2a, in accordance with an embodiment of the present invention;

FIG. 3a is a partially cut-out perspective of an exemplary device primarily targeted for increasing girth of a penis, in accordance with an embodiment of the present invention;

FIG. 3b is a cross section view of the device of FIG. 3a, in accordance with an embodiment of the present invention;

FIG. 4a is a partially cut-out perspective of an exemplary device targeted for increasing girth of a penis and also significantly reducing the stimulation, in accordance with an embodiment of the present invention;

FIG. 4b is a cross section view of the device of FIG. 4a, in accordance with an embodiment of the present invention;

FIG. 4c is a partially cut-out perspective of another exemplary device targeted for increasing the girth of penis but offers much higher stimulation, in accordance with an embodiment of the present invention;

FIG. 4d is a cross section view of the device of FIG. 4c, in accordance with an embodiment of the present invention;

FIG. 5a is a partially cut-out perspective view of another exemplary device primarily targeted for increasing both girth and length of a penis, in accordance with an embodiment of the present invention;

FIG. 5b is a cross section view of the device of FIG. 5a, in accordance with an embodiment of the present invention;

FIG. 5c is a partially cut-out perspective view of another exemplary device similar to the device of FIG. 5a but exhibits a much higher increase in girth and length of a penis, in accordance with an embodiment of the present invention;

FIG. 5d is a cross section view of the device of FIG. 5d, in accordance with an embodiment of the present invention;

FIG. 6a is a partially cut-out perspective view of an exemplary device targeting PME/girth enhancement, illustrating a partial rolled up position, in accordance with an embodiment of the present invention;

FIG. 6b is a front elevation view of the device of FIG. 6a in a fully rolled up position, in accordance with an embodiment of the present invention;

FIG. 6c is a top elevation view of the device of FIG. 6a in a fully rolled up position, in accordance with an embodiment of the present invention;

FIG. 6d is a partially cut-out perspective view of an exemplary device for targeting girth and length enhancement, illustrating the partial rolled up position, in accordance with an embodiment of the present invention;

FIG. 7 is a partially cut-out perspective view of an exemplary device including circumferential reinforcement bands, in accordance with an embodiment of the present invention;

FIG. 8a is a perspective view of another exemplary device to enable girth enhancement, a portion of the device being devoid of a wall, in accordance with an embodiment of the present invention;

FIG. 8b is a partially cut out perspective view of the device of FIG. 8a, in accordance with an embodiment of the present invention;

FIG. 8c is a perspective view of another exemplary device to enable both girth and length enhancement, a portion of the device being devoid of a wall, in accordance with an embodiment of the present invention;

FIG. 8d is a partially cut out perspective view of the device of FIG. 8c, in accordance with an embodiment of the present invention;

FIG. 9a is a perspective view of another exemplary device in form of a sleeve and targeted for increasing girth of a penis, in accordance with an embodiment of the present invention;

FIG. 9b is a partially cut-out perspective of the device of FIG. 9a, in accordance with an embodiment of the present invention;

FIG. 9c is a perspective view of another exemplary device in form of a sleeve and targeted for increasing both girth and length of a penis, in accordance with an embodiment of the present invention;

FIG. 9d is a partially cut-out perspective of the device of FIG. 9c, in accordance with an embodiment of the present invention;

FIG. 10a is a perspective view of an exemplary device in form of a sleeve having an open top portion, in accordance with an embodiment of the present invention;

FIG. 10b is a partially cut-out perspective of the device of FIG. 10a, in accordance with an embodiment of the present invention;

FIG. 11a is a partially cut-out perspective view of an exemplary device including a shape retaining inner reinforcement material, in accordance with an embodiment of the present invention;

FIG. 11b is a perspective view of the device of the shape retaining inner reinforcement material used in FIG. 11a, in accordance with an embodiment of the present invention;

FIG. 11c is a cross sectional view of the device of FIG. 11a along plane M1-M1, in accordance with an embodiment of the present invention;

FIG. 12a is a perspective view of another exemplary device that includes a flange for mounting additional mechanisms, in accordance with an embodiment of the present invention;

FIG. 12b is a partially cut-out perspective of the device of FIG. 12a, in accordance with an embodiment of the present invention;

FIG. 13a is a perspective view of an accessory that can be slid over an exemplary device of the present invention, in accordance with an embodiment of the present invention;

FIG. 13b is a perspective view of another exemplary device including the accessory of FIG. 13a, in accordance with an embodiment of the present invention;

FIG. 13c is a vertical cross sectional view of the device of FIG. 13b, in accordance with an embodiment of the present invention;

FIG. 14a is a perspective view of an exemplary device in form of a condom that is designed to enhance girth, in accordance with an embodiment of the present invention;

FIG. 14b is a longitudinal cross sectional view of the device of FIG. 14a, in accordance with an embodiment of the present invention; and

FIG. 14c is a cross sectional view of the device of FIG. 14a, in accordance with an embodiment of the present invention.

Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention.

There may be additional structures described in the foregoing application that are not depicted on one of the described drawings. In the event such a structure is described, but not depicted in a drawing, the absence of such a drawing should not be considered as an omission of such design from the specification.

### SUMMARY

The present invention provides a tubular device for being disposed on a penis of a user. The tubular device includes a proximal end to facilitate disposition of the tubular device on the penis and a distal end substantially opposite the proximal end. Further, the tubular device includes a wall defining a hollow tubular body for receiving the penis. The hollow tubular body extends between the proximal end and the distal end. Furthermore, the wall includes at least a first set of regions having a first wall thickness and a second set of regions having a second wall thickness, such that the first wall thickness is greater than the second wall thickness and that at least one of an underside of the penis and underside of glans of the penis is not covered by the first set of regions or a portion thereof. The tubular device facilitates at least one of an effective increase in a girth of the penis, an effective increase in a length of the penis and an effective increase in time to ejaculation for the user of the device.

In some embodiments, the first wall thickness of the tubular device is 15 mm or less and the second wall thickness of the tubular device is 0.8 mm or less and thinner than the first wall thickness.

In some embodiments, the first set of regions cover 35% to 80% of the wall and the second set of regions cover 20% to 50% of the wall.

Further, in some embodiments, the proximal end is open and the distal end is closed, allowing the penis to be covered substantially completely.

Further, in some embodiments, both the proximal end and the distal end are open leaving glans of the penis substantially uncovered.

In some other embodiments, at least a portion of the tubular device is devoid of the wall, thereby leaving a portion of the penis uncovered to experience uninhibited stimulation.

In some embodiments, the tubular device also includes one or more bands over the wall to secure the tubular device over the penis.

In some embodiments, the hollow tubular body of the tubular device further includes a reservoir for accumulating semen released from the penis. Further, the reservoir is disposed at the distal end and is connected to the hollow tubular body via a duct.

In some embodiments, the tubular device includes a flange at the proximal end, such that the flange allows mounting of additional devices for enhanced clitoral or vaginal stimulation.

In some embodiments, the tubular device is rolled up from the proximal end for being packaged.

In some embodiments, a portion of an outer surface of the wall includes perforations and patterns for providing enhanced vaginal stimulation.

In some other embodiments, a de-sensitizing liquid is applied on a portion of an inner surface of the wall for delaying ejaculation for the user of the device.

In some other embodiments, a vaginal stimulation chemical is applied on a portion of an outer surface of the wall for providing enhanced vaginal stimulation.

In some embodiments, the tubular device also includes a shape retaining inner reinforcement material placed between an inner surface and an outer surface of the wall.

In some embodiments, the present invention provides a tubular device for being disposed on a penis of a user. The tubular device includes a proximal end to facilitate disposition of the tubular device on the penis and a distal end substantially opposite the proximal end. Further, the tubular device includes a wall defining a hollow tubular body for receiving the penis. The hollow tubular body extends between the proximal end and the distal end. Furthermore, the wall includes at least a first set of regions having a first wall thickness and a second set of regions having a second wall thickness, such that the first wall thickness is greater than the second wall thickness and that at least one of an underside of the penis and underside of glans of the penis is not covered by the first set of regions or a portion thereof. This design of the tubular device facilitates an effective increase in a girth of the penis and an effective increase in a length of the penis and an effective increase in time to ejaculation for the user of the device. Further, at the same time providing maximum sensory perception to the user as the maximum sensory perception is received on stimulation of the underside of the penis and the underside of glans of the penis, which according to the present invention is not covered by the first set of regions, i.e. the underside of the penis and the underside of glans of the penis are either covered by the second set of regions or are devoid of any wall.

### DESCRIPTION OF THE EXEMPLARY EMBODIMENTS

Before describing the present invention in detail, it should be observed that the present invention utilizes a combination of method steps and apparatus components related to a method of manufacturing a semiconductor device. Accordingly the apparatus components and the method steps have been represented where appropriate by conventional symbols in the drawings, showing only specific details that are pertinent for an understanding of the present invention so as not to obscure the disclosure with details that will be readily apparent to those with ordinary skill in the art having the benefit of the description herein.

While the specification concludes with the claims defining the features of the invention that are regarded as novel, it is believed that the invention will be better understood from a consideration of the following description in conjunction with the drawings, in which like reference numerals are carried forward.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The terms “a” or “an”, as used herein, are defined as one or more than one. The term “another”, as used herein, is defined as at least a second or more. The terms “including” and/or “having” as used herein, are defined as comprising (i.e. open transition). The term “coupled” or “operatively coupled” as used herein, is defined as connected, although not necessarily directly, and not necessarily mechanically.

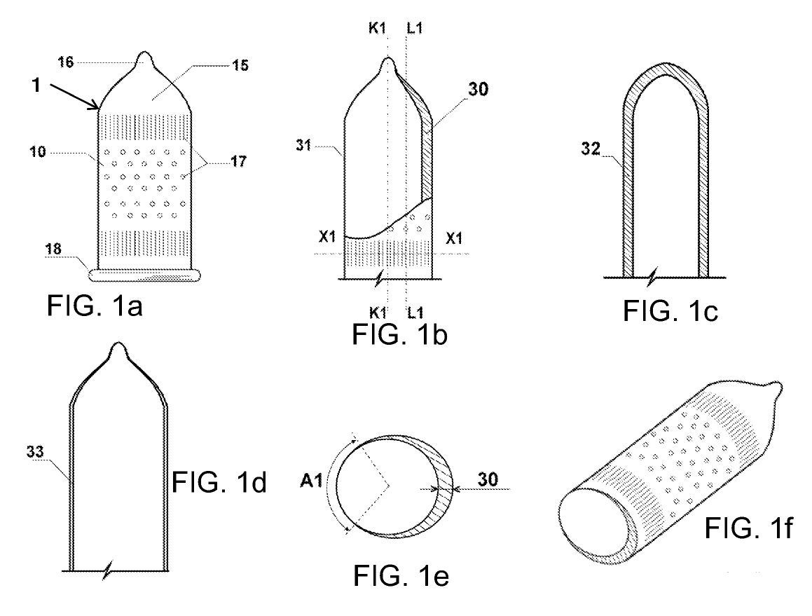

Referring now to the drawings FIG. 1a illustrates a top elevation view of an exemplary device 1, in accordance with an embodiment of the present invention. The device 1 in this embodiment is in form that can be used by an user in a manner similar to using a condom. The device 1 includes a hollow tubular body 10, a wall 15, a proximal end 18 and a distal end 16. In an embodiment, the device 1 may also include patterns 17 on an outer surface of the wall 15.

FIG. 1b illustrates a partially cut-out perspective view of the exemplary tubular device 1. The tubular device 1 may be referred to as the device 1 in the description of the present invention. The figure illustrates a thick wall 30 on one side and a thin wall 31 on a diametrically opposite side. This variation in thickness around periphery is further illustrated in FIGS. 1e and 1f, which is a cross-sectional view of the device 1 along the plane X1-X1. As can be seen, area under A1 illustrates a thin wall section of the device 1. The figure illustrates how the thick wall 30 on one side of the device 1 gradually thins and becomes a thin wall 31 on a diametrically opposite side of the device 1.

Varying degree of wall thickness along length of the device 1 is further illustrated in FIG. 1c and FIG. 1d. For example, FIG. 1c is a longitudinal cross sectional view of the device 1 along the plane L1-L1, illustrating a relatively large wall thickness 32 at this section of the device 1 and FIG. 1d is a longitudinal cross sectional view of the device 1 along the plane K1-K1, illustrating a reduced wall thickness 33 at this section of the device 1.

The thick wall 30 offers restraining of stimulation to a penis of the user during intercourse. The degree of this restraining of stimulation can be increased by increasing area covered with the thick wall 30 and also by increasing wall thickness. Similarly for relatively higher degree of stimulation; the wall thickness can be reduced; also area covered by the thicker wall 30 can be reduced. Various embodiments are thus possible with a variety of wall thickness and with variation in amount of area covered by the thick wall 30. These embodiments can be accordingly be used for desired amount of restraining of stimulation. In addition a variety of colors, textures (like the patterns 17) and appropriate construction materials can be leveraged for optimal design and durability under any usage condition.

The illustrations used herein are not intended to be limiting but rather to provide an understandable description of the invention. Therefore, those ordinarily skilled in the art will appreciate that the wall 15 can have a first wall thickness over a first set of regions and a second wall thickness over a second set of regions as long as the underside of a penis and underside of glans of the penis is not covered by the first set of regions or a portion thereof. Further, the first set of regions and the second set of regions need not be continuous and can be discontinuous. Also, it should be appreciated that a portion of the device 1 may be completely devoid of a wall. This design of the tubular device facilitates an effective increase in a girth of the penis and an effective increase in a length of the penis and an effective increase in time to ejaculation for the user of the device. Further, at the same time providing maximum sensory perception to the user as the maximum sensory perception is received on stimulation of the underside of the penis and the underside of glans of the penis, which according to the present invention is not covered by the thick wall 30, but are either covered by the thin wall 31 or are completely devoid of any wall to maintain providing maximum sensory perception to the user.

In an embodiment, the device 1 has a first wall thickness 31 of 15 mm or less and the second wall thickness 30 of 0.8 mm or less. Further, the second wall thickness 30 is thinner than the first wall thickness 31. Also, in the device 1, the first set of regions cover 35% to 80% of the wall 15 and the second set of regions cover 20% to 50% of the wall 15.

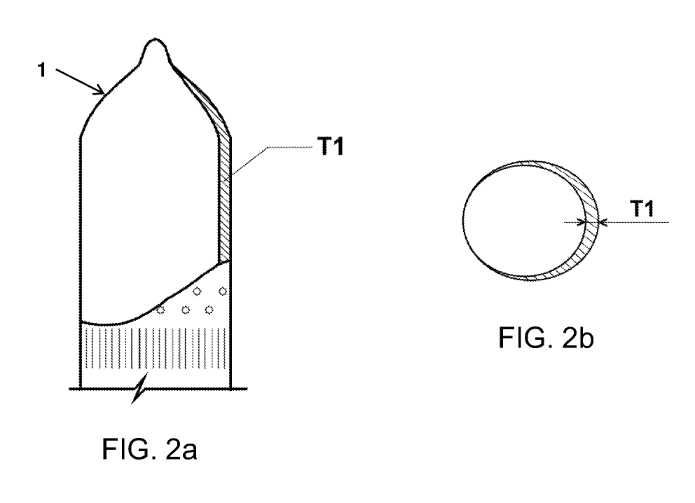

Moving on FIG. 2a illustrates a partially cut-out perspective of the device 1 in an embodiment that is primarily targeted for addressing pre mature ejaculation (PME). In this embodiment the device 1 has a first wall thickness T1 and a second wall thickness which is same as that of a typical condom. A cross-sectional view of the device in this embodiment can be seen in FIG. 2b. A typical wall thickness T1 for embodiments targeting PME will be preferably less than 3 mm but can be higher for certain design variations.

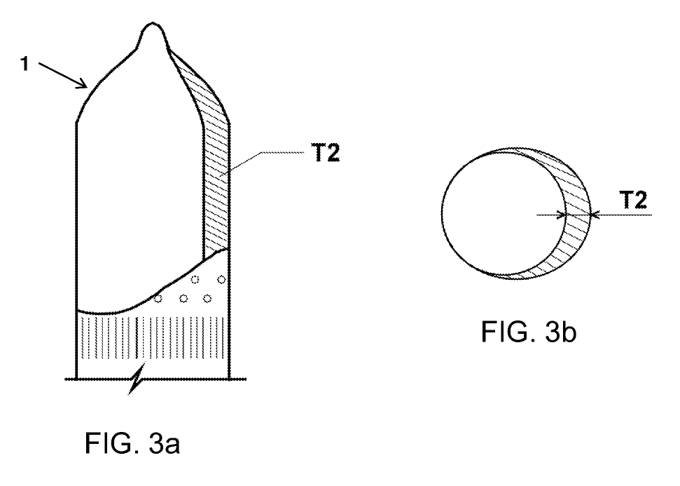

FIG. 3a is a partially cut-out perspective view of the device 1 according to another embodiment of the present invention. This embodiment is a variant of the device 1 in FIG. 2a and is primarily targeted for increasing girth of the penis by adding thickness to one side of the device 1 while keeping a very thin wall on remaining portion of the device 1. This permits sufficient stimulation to the penis and at the same time increase the effective girth of the penis. The wall thickness T2 in this embodiment is much larger than T1 in FIG. 2a.

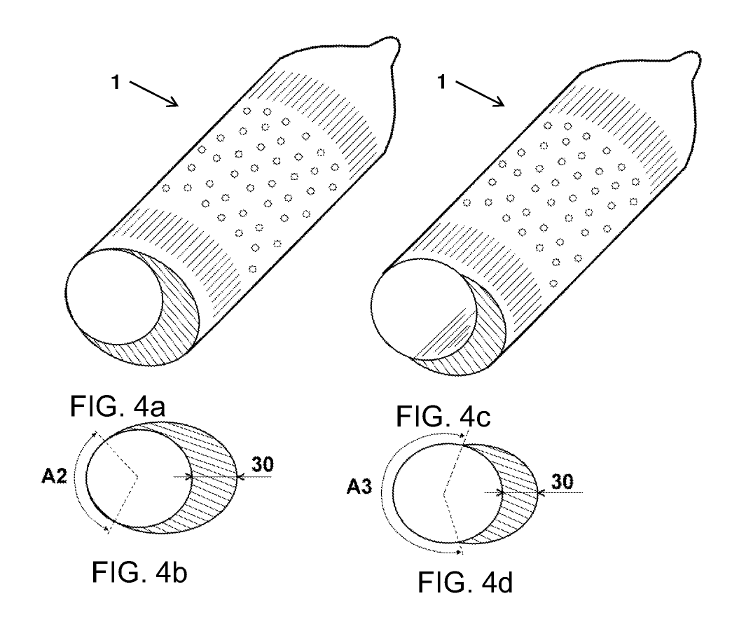

FIGS. 4a and 4b illustrate another embodiment of the device 1. In this embodiment, the device 1 is a variant to a typical condom, but has the thick wall 30 over a portion of the device 1. Further, the thick wall 30 covers most of the wall of the device 1. This embodiment causes a significant increase in girth and offers significant reduction is stimulation to male partner.

A variation of the embodiment described in FIGS. 4a and 4b can be seen in FIGS. 4c and 4d. The FIGS. 4c and 4d illustrate the device 1 having the thick wall 30 over a portion of the wall. This embodiment also enables significant increase in girth but when compared to the embodiment in FIG. 4a, offers higher stimulation to the male partner. This is achieved as area covered with a thin wall A3 is much larger to corresponding area A2 in FIG. 4a

Larger the area covered by the thin wall, greater will be the stimulation experienced by the user. Therefore, it should be apparent to those ordinarily skilled in the art that this principle can be leveraged appropriately for a number of embodiments offering varying degree of stimulation and girth enhancement.

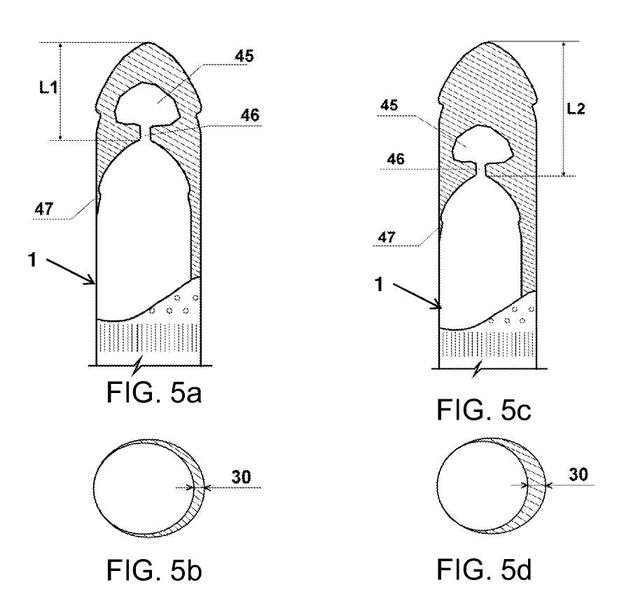

Moving on to FIGS. 5a and 5b, there is illustrated another embodiment of the device 1, in accordance with the present invention. This embodiment can also be a variant to a typical condom construction but has a thick wall 30 over a portion of the device 1. This embodiment enables an enhancement in the length of the penis. Extension L1 on the distal end of the device 1 adds to a length of the penis of the user wearing the device 1. Further, a cavity 45 has been provided. The cavity 45 acts as a reservoir for accumulating semen released by the penis of the user. A reservoir duct 46 for connecting the cavity 45 to the hollow tubular body has also been provided. The cavity 45 is beneficial in reducing risk of rupture of the thin wall section of the device 1. The extension L1 can be of various lengths depending upon the requirement of the user. Another example, with a longer extension L2 has been provided in FIGS. 5c and 5d. The length of the extension L2 in FIG. 5c is larger than that of the extension L1. Also wall thickness in FIG. 5d is larger than that of corresponding wall thickness in FIG. 5b. Therefore, it should be appreciated that a range of length as well as girth extension is feasible in appropriate embodiments according to the present invention.

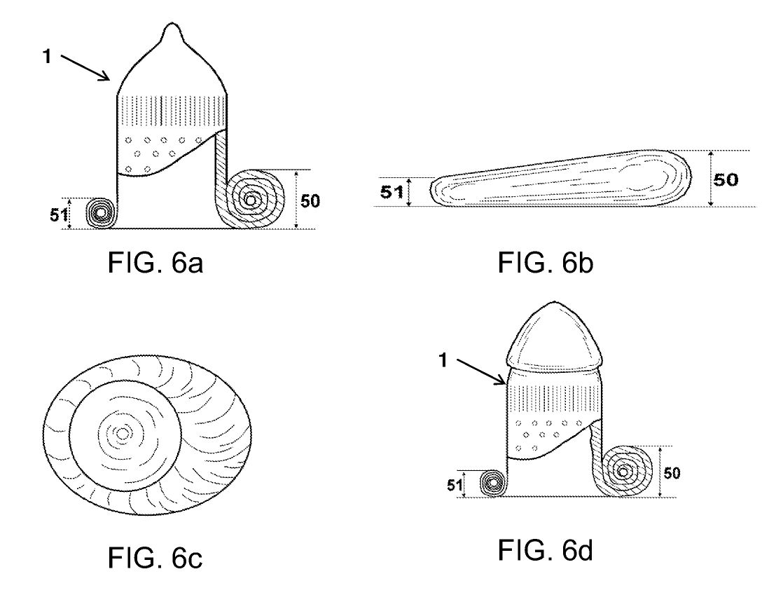

FIG. 6a is a partially cut-out perspective view of an embodiment of the device 1 for targeting PME/girth enhancement. The FIG. 6a illustrates the device in a partial rolled up position. Sides because of the variation in thickness of the wall will roll up differently, for example, a side is shown to have a rolled up thickness 50 which is greater than a rolled up thickness 51 on opposite side.

FIGS. 6b and 6c show other views of the device 1 according to this embodiment. Further, FIG. 6d illustrates the device 1 in an embodiment for targeting girth and length enhancement illustrating a partial rolled up position.

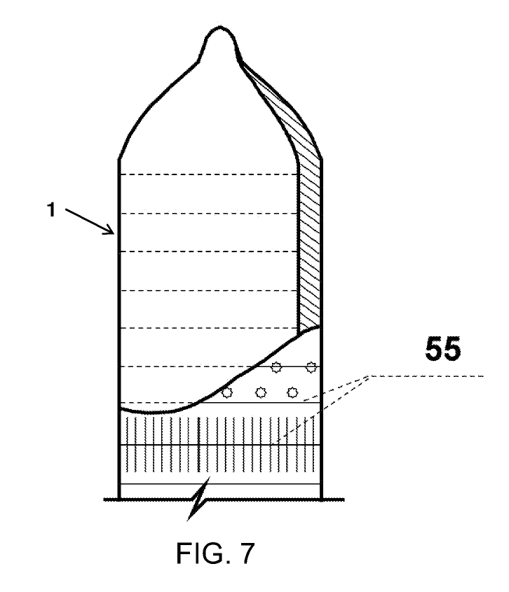

FIG. 7 is a partially cut-out perspective view of the device 1 in accordance with another embodiment of the present invention. In this embodiment the device 1 is shown to have circumferential reinforcement bands 55 that enable the device 1 to fit better/grip onto the penis of the user. The reinforcement bands 55 also distribute weight of the wall with a higher wall thickness in more uniform manner. The reinforcement bands 55 may appear as circumferential rings or any other suitable pattern and can also provide enhanced stimulation to a female partner by providing desired friction. This feature may be leveraged for any other embodiment of the device 1. Further, in another embodiment, the device 1 may be provided with specialized features/materials on an inner surface of the device 1 to ensure better fit as well as minimize chances of slip-offs.

All the embodiments illustrated so far from FIG. 1 to FIG. 7 are variants of a typical condom having a larger wall thickness on a portion or having a length extension or both girth and length extensions. However, each of these can be rolled as illustrated. This ability to rollup also enables application of the device in a manner similar to wearing a condom and makes adoption easy. It also enables creation of more acceptable packaging as well as marketing plans.

However, depending on the wall thickness; rolling it up like a traditional condom may not be feasible beyond a certain wall thickness. Such embodiments may take the form of a sleeve instead of a traditional condom. Therefore, all the embodiments from FIG. 1 to FIG. 7 can also be created as sleeves. Following part of the description will further illustrate the embodiments of the present invention where the device will be described in the form of a variant of a typical sleeve.

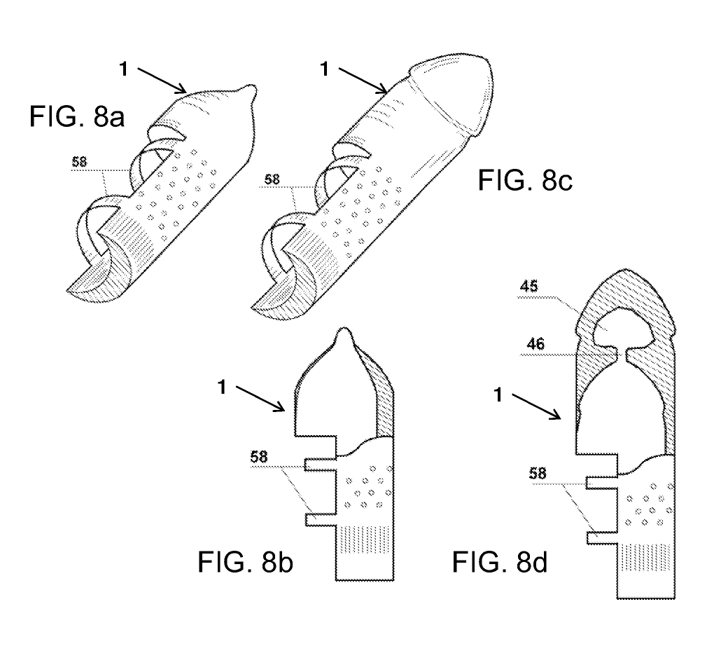

FIGS. 8a and 8b illustrate an embodiment in which the device 1 is in form of a sleeve. The device 1 in this embodiment is designed such that it can enable girth enhancements. The device 1 covers only a portion of the penis, leaving remaining area on underside of the penis and glans uncovered to experience normal stimulation during sexual intercourse. The device 1 is also shown to include two bands 58 to secure the device 1 over the penis. It should be appreciated that the number of bands 58 can be more or less than those depicted in the figure.

Embodiments in FIGS. 8c and 8d illustrate the device 1 that can enable girth as well as length enhancements. The length enhancement mechanism is similar to the one discussed earlier in FIGS. 5a and 5c. The need for a reservoir and a reservoir duct is optional in embodiments that are based on sleeve design.

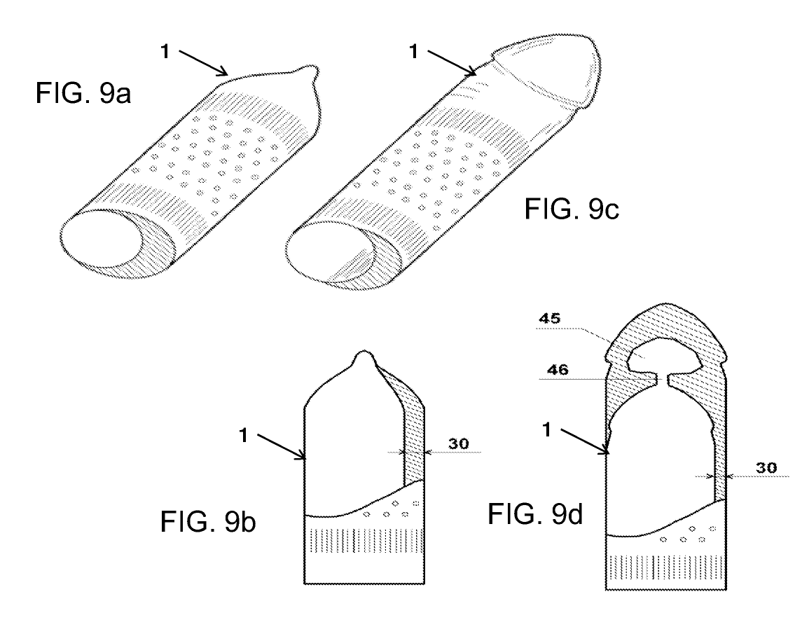

FIGS. 9a and 9b illustrate another embodiment of the device 1 in the form of a sleeve that is primarily targeted for increasing girth of penis by adding thickness to one side of the penis while leaving a very thin wall on remaining portion of the device 1, thereby permitting sufficient stimulation to penis. Similarly, FIGS. 9c and 9d illustrate another embodiment of the device 1 in form of a sleeve that is primarily targeted for increasing both girth and length of the penis.

The embodiment in the form of a sleeve can support much larger girth enhancements along with length enhancement than the embodiments where the device is in form of a condom.

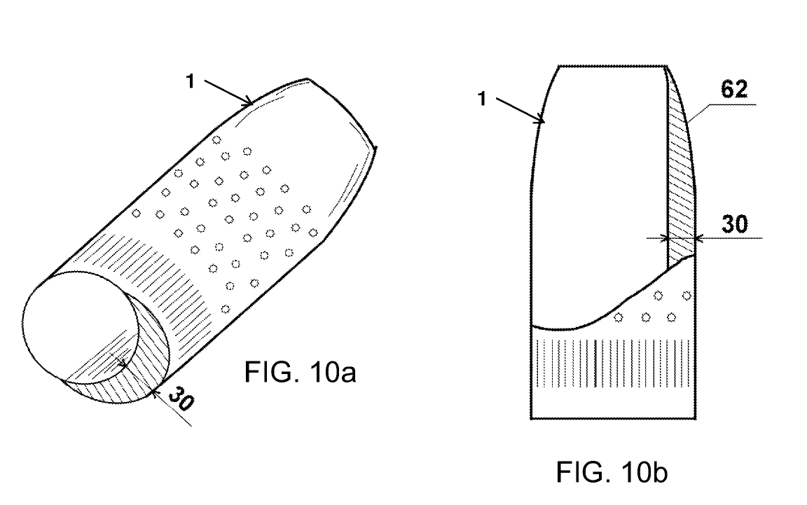

FIGS. 10a and 10b illustrate another embodiment of the device 1 in form of a sleeve. The device in this embodiment permits controlled stimulation and also increasing girth, while allowing sufficient stimulation at the same time. The device 1 may come in a variety of maximum thickness to facilitate increased penis girth. Further the device 1 is shown to have an open top portion 62 that allows glans to experience maximum stimulation.

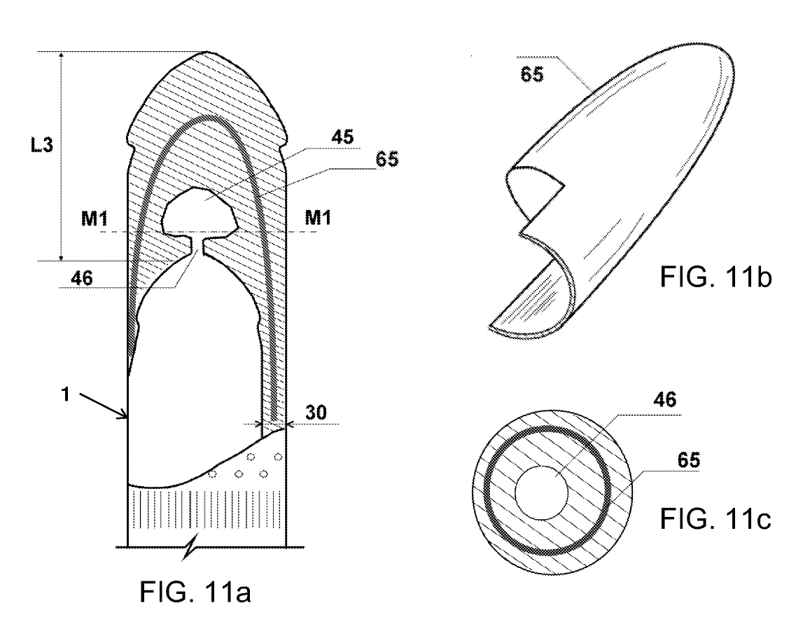

FIG. 11a illustrates another embodiment of the device 1. The device 1 in this embodiment is provided with an increased girth as well as length of the penis. Additionally, a shape retaining inner reinforcement material 65 is provided to the device 1. The purpose of this shape retaining inner reinforcement material 65 is to provide rigidity to length extension of the device 1. The shape retaining inner reinforcement material 65 can be made of a different material than that of the device 1. The material can have a higher rigidity and shape retaining properties. Further as illustrated the shape retaining inner reinforcement material 65 is completely surrounded by a softer material of the device 1. This design and construction of the device 1 may offer support during any short loss of erection.

It should be apparent to those ordinarily skilled in the art that all embodiments that are in shape of a sleeve can come in various shapes and sizes and also include different perforations or patterns in area covered with thin wall for enhanced stimulation experience.

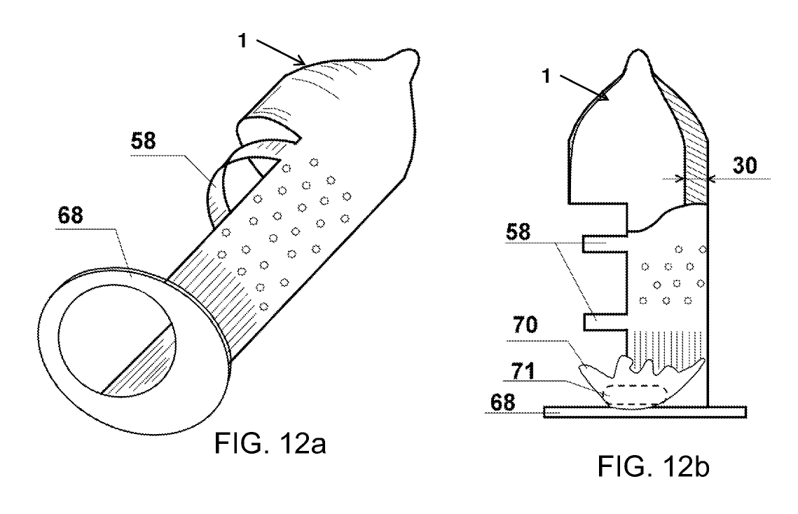

Moving on, FIGS. 12a and 12b illustrate another embodiment of the device 1 that can be custom made to match exact form (shape/size) of the user's penis. According to this embodiment, the device 1 can support much larger girth enhancements along with length enhancements. Further, the device 1 in this embodiment is shown to include a flange 68 at the proximal end to permit mounting of additional stimulating mechanisms including vibratory mechanisms. FIG. 12b also illustrates the flange 68 at the proximal end and an integral clitoral stimulating formation 70 that has a cavity 71 to accept a vibrating mechanism for enhanced sexual experience.

The exact dimensions for customizing the device 1 may be received online or a mould may be shipped from the user for making the device 1 to suit exact size of the user.

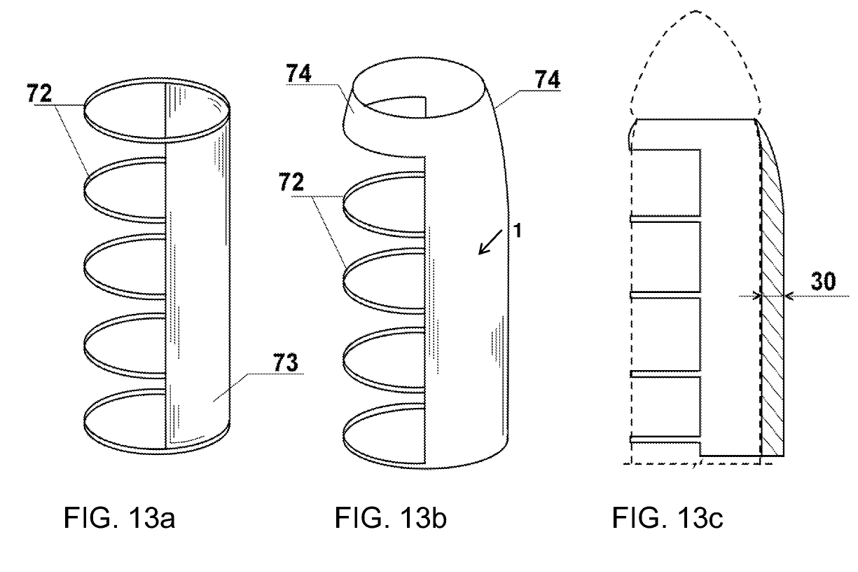

FIG. 13a illustrate an accessory 73 in form of a thin tubular construction that can be slid over various embodiments of the device 1 to enable better hold. The accessory 73 has a number of elastic band like formations 72 of suitable elasticity to enable a comfortable yet appropriate force to hold the device 1 in place during intercourse. It should be appreciated that the accessory can work for a range of girths of the device 1. FIGS. 13b and 13c illustrate a view in which the accessory 73 has been applied on the device 1. The device 1 can have a tapered distal end 74 for reducing initial friction. Similar variants of this design with an open distal end exposes entire glans and a significant portion of the penis, thereby offering enhanced stimulation to the user while enabling girth enhancement at the same time.

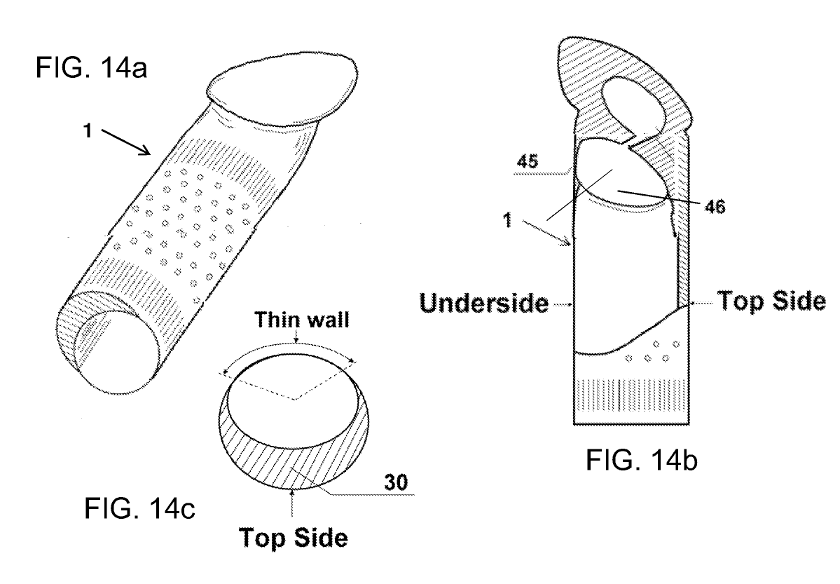

Moving on to FIGS. 14a, 14b and 14c illustrate another embodiment of the device 1 which is a variant of condom type construction and is designed to cover underside of the penis with a thinner wall thickness and topside with a thicker wall thickness, thereby enhancing the girth and at the same time maintaining stimulation. The embodiment illustrated in this figure also has an extension for enhancing length of the penis.

This design of the tubular device 1 facilitates an effective increase in a girth of the penis and an effective increase in a length of the penis and an effective increase in time to ejaculation for the user of the device. Further, at the same time providing maximum sensory perception to the user as the maximum sensory perception is received on stimulation of the underside of the penis and the underside of glans of the penis, which according to the present invention is not covered by the thick wall 30, but are either covered by the thin wall or in another embodiment may be completely devoid of any wall to maintain providing maximum sensory perception to the user.

Further, in the figures a transition from maximum to minimum wall thickness of the device 1 has been shown to be gradual but it should be appreciated that this transition can also be abrupt.

Further, the device in accordance with the present invention can be manufactured or cut to user specific sizes for best possible fit. Data relating to the size/shape of the penis of the user may be collected from the user using various online or offline means. Further, such information can be calculated using size information from manual or machine based measurements. Also, such information can be obtained by receiving a mould from the user in order to manufacture appropriate user specific sizes.

Also, the device in accordance with the present invention can be provided with enhanced elasticity ranges, higher rupture strengths and special sizes. Further, in cases of the device which is in the form of a sleeve, a typical condom can be worn over the device according to the embodiments of the present invention to offer birth control protection and enhanced fit.

Embodiments of the present invention may use thicker walls on more than one continuous region on a periphery of the device for a range of lengths. As an example an embodiment may have thin wall regions approximately 1-3 cm wide on two opposite faces and just one thick wall region on the remaining portion of the device.

In other embodiments, the device may also be provided by means of securing the device more firmly by having elastic bands attached to the proximal end that can loop over testicles of the user.

The device may also have a de-sensitizing material applied on an inner surface of the wall of the device such that the device is provided with additional mechanisms for climax control during sexual intercourse. In another embodiment, of the invention the de-sensitizing material may be separately packaging and the user may apply the de-sensitizing material inside the device's reservoir tip or directly onto penis's glans prior to wearing the device.

In another embodiment, a de-sensitizing material in a viscous/semi solid state that turns to liquid form after coming into the contact with human body temperature may be used.

Similarly a vaginal stimulation chemical/gel/liquid/powder may be prepackaged separately and may be applied by the user, on an outer surface of the device after the device has been put onto penis of the user.

The overall approach therefore is to strike a balance wherein stimulation to the user of the device is restrained to an acceptable degree and vaginal stimulation is enhanced so that the female partner can achieve orgasm sooner than normal, where as the user is able to prolong the time to ejaculation or orgasm.

Various embodiments, as described above, provide a device for being disposed on a penis of a user in order to facilitate at least one of an effective increase in a girth of said penis, an effective increase in a length of said penis and an effective increase in time to ejaculation for said user of said device. The device as described in the present invention has several advantages. One of the several advantages is that the device of the present invention provides targeted sexual stimulation restraining effect to male partner during sexual intercourse to address condition of PME. Further, the device instantaneously provides an increase in an effective girth and length of the penis of the user. Additionally, the device also provides enhanced stimulation to the female partner during sexual intercourse. Also, the design of the present device ensures that a balance between reducing stimulation to the male partner and enhancing vaginal stimulation of the female partner is achieved, such that parity can be brought in attaining climax of sexual intercourse for both the male and the female partner.

Further, another advantage provided by the device is that almost every intended user is familiar with using a condom which is how the present device can be applied. Further, the ease of availability & mass marketing feasibility that is provided by the device because of its socially acceptable form. Further, the device can be available off the shelves in supermarkets or be sold at online retailer in the same form as a typical condom is, thereby turning it into an acceptable average product in comparison to adult only product/device available today. Additionally, availability in a variety of sizes to enable best fit to ensure high customer satisfaction is another added advantage of the present invention.

While the invention has been disclosed in connection with the preferred embodiments shown and described in detail, various modifications and improvements thereon will become readily apparent to those ordinarily skilled in the art. Accordingly, the spirit and scope of the present invention is not to be limited by the foregoing examples, but is to be understood in the broadest sense allowable by law.

All documents referenced herein are hereby incorporated by reference.

What is claimed is:

1. A tubular device for being disposed on a penis of a user, said tubular device comprising:
a proximal end to facilitate disposition of said tubular device on said penis;
a distal end substantially opposite said proximal end; and
a wall defining a hollow tubular body for receiving said penis, said hollow tubular body extending between said proximal end and said distal end, said wall comprising at least:
a first set of regions having a first wall thickness; and
a second set of regions having a second wall thickness, wherein said first wall thickness is greater than said second wall thickness, further wherein at least one of an underside of said penis and underside of glans of said penis is not covered by said first set of regions or a portion thereof;
whereby said tubular device facilitates at least one of an effective increase in a girth of said penis, an effective increase in a length of said penis and an effective increase in time to ejaculation for said user of said device.

2. The tubular device according to claim 1, wherein said first wall thickness is 15 mm or less and said second wall thickness is 0.8 mm or less and thinner than said first wall thickness.

3. The tubular device according to claim 1, wherein said first set of regions cover 35% to 80% of said wall and said second set of regions covers 20% to 50% of said wall.

4. The tubular device according to claim 1, wherein said proximal end is open and said distal end is closed, thereby covering said penis substantially completely.

5. The tubular device according to claim 1, wherein both said proximal end and said distal end are open, thereby leaving glans of said penis substantially uncovered.

6. The tubular device according to claim 1, wherein at least a portion of said tubular device is devoid of said wall, thereby leaving a portion of said penis uncovered to experience uninhibited stimulation.

7. The tubular device according to claim 1, wherein said tubular device further comprises one or more bands over said wall to secure said tubular device over said penis.

8. The tubular device according to claim 1, wherein said hollow tubular body further comprises a reservoir for accumulating semen released from said penis, said reservoir being disposed at said distal end and being connected to said hollow tubular body via a duct.

9. The tubular device according to claim 1, wherein said tubular device comprises a flange at said proximal end, said flange allows mounting of additional devices for enhanced clitoral or vaginal stimulation.

10. The tubular device according to claim 1, wherein said device is rolled up from said proximal end for being packaged.

11. The tubular device according to claim 1, wherein a portion of an outer surface of said wall comprises perforations and patterns for providing enhanced vaginal stimulation.

12. The tubular device according to claim 1, wherein a de-sensitizing liquid is applied on a portion of an inner surface of said wall for delaying ejaculation for said user of said device.

13. The tubular device according to claim 1, wherein a vaginal stimulation chemical is applied on a portion of an outer surface of said wall for providing enhanced vaginal stimulation.

14. The tubular device according to claim 1, wherein said tubular device comprises a shape retaining inner reinforcement material between an inner surface and an outer surface of said wall.
